SEQUENCE LISTING (11 sequences)

SEQ ID NO: 1 - protein, 173 aa, Artificial Sequence
  Definition of Artificial Sequence Synthetic polypeptide
MGPKKKRKVIMNTKYNKEFLLYLAGFVDGDGSIIASIRPRQSCKFKHELELRFQVTQKTQRRWFLDKLVDEIGVGYVRDRGSVSDYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - DNA, 22 bp, Artificial Sequence
  Definition of Artificial Sequence Synthetic oligonucleotide
ctccgggtcgtacgacccggag

SEQ ID NO: 3 - DNA, 22 bp, Artificial Sequence
  Definition of Artificial Sequence Synthetic oligonucleotide
ctccgggtcgtacgacccggag

SEQ ID NO: 4 - protein, 360 aa, Artificial Sequence
  Definition of Artificial Sequence Synthetic polypeptide
MGPKKKRKVIMNTKYNKEFLLYLAGFVDGDGSIKAQIRPRQSRKFKHELELTFQVTQKTQRRWFLDKLVDEIGVGKVYDRGSVSDYELSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGATKSKEFLLYLAGFVDGDGSIIASIRPRQSCKFKHELELRFQVTQKTQRRWFLDKLVDEIGVGYVRDRGSVSDYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 5 - DNA, 22 bp, Artificial Sequence
  Definition of Artificial Sequence Synthetic oligonucleotide
tgcctcctctagagacccggag

SEQ ID NO: 6 - DNA, 22 bp, Artificial Sequence
  Definition of Artificial Sequence Synthetic oligonucleotide
ctccgggtctctagaggaggca

SEQ ID NO: 7 - DNA, 522 bp, Artificial Sequence
  Definition of Artificial Sequence Synthetic polynucleotide
atgggcccgaagaagaagcgcaaggtcatcatgaacaccaagtacaacaaggagttcctgctctacctggcgggcttcgtggacggggacggctccatcatcgcctccatccgcccgcgtcagtcctgcaagttcaagcatgagctggaactccggttccaggtcacgcagaagacacagcgccgttggttcctcgacaagctggtggacgagatcggggtgggctacgtgcgcgaccgcggcagcgtctccgactaccgcctgagccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgagaccgtccgcgccgtgctggacagtctctccgagaagaagaagtcgtccccctag

SEQ ID NO: 8 - DNA, 1083 bp, Artificial Sequence
  Definition of Artificial Sequence Synthetic polynucleotide
atgggcccgaagaagaagcgcaaggtcatcatgaacaccaagtacaacaaggagttcctgctctacctggccggcttcgtggacggcgacggctccatcaaggcgcagatccgtccgcggcagagccggaagttcaagcacgagctcgagctgaccttccaggtgacccagaagacgcagaggcgctggttcctcgacaagctggtggacgagatcggggtgggcaaggtctacgaccgcgggtcggtgtccgactacgagctctcccagatcaagcccctgcacaacttcctcacccagctccagccgttcctgaagctcaagcagaagcaggccaacctcgtgctgaagatcatcgagcagctgccctccgccaaggaatccccggacaagttcctggaggtgtgcacgtgggtggaccagatcgcggccctcaacgacagcaagacccgcaagacgacctcggagacggtgcgggcggtcctggactccctcccaggatccgtgggaggtctatcgccatctcaggcatccagcgccgcatcctcggcttcctcaagcccgggttcagggatctccgaagcactcagagctggagcaactaagtccaaggaattcctgctctacctggcgggcttcgtggacggggacggctccatcatcgcctccatccgcccgcgtcagtcctgcaagttcaagcatgagctggaactccggttccaggtcacgcagaagacacagcgccgttggttcctcgacaagctggtggacgagatcggggtgggctacgtgcgcgaccgcggcagcgtctccgactaccgcctgagccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgagaccgtccgcgccgtgctggacagtctctccgagaagaagaagtcgtccccctag

SEQ ID NO: 9 - protein, 38 aa, Artificial Sequence
  Definition of Artificial Sequence Synthetic polypeptide
PGSVGGLSPSQASSAASSASSSPGSGISEALRAGATKS

SEQ ID NO: 10 - protein, 9 aa, Artificial Sequence
  Definition of Artificial Sequence Synthetic peptide
MAPKKKRKV

SEQ ID NO: 11 - DNA, 1822 bp, Arabidopsis thaliana
ccttgacgaattctatatacctagttcgttttttcttcctcaaatatatctttttcaatttatttggtttttctttgggtgcaacttcacctcacaaaattttctctctttttttatattaatttgagttaggcctttttgatttcatagatgagtcgtctagtcgtctggatttgatgtggttatagtcttacagagacctttgattgaaataagaacaaaagcaagaatacatacatcctcttcatcttacacccatccttttttatttttctagggttttatttttttttaatttattttttttctttgatttttatattctctctctctctcaaatctttactcatctgggtatggaagaataccagcatgacaacagcaccactcctcaaagagtaagtttcttgtactctccaatctcttcttccaacaaaaacgataacacaagtgataccaacaacaacaacaacaataataatagtagcaattatggtcctggttacaataatactaacaacaacaatcatcaccaccaacacatgttgtttccacatatgagctctcttctccctcaaacaaccgagaattgcttccgatctgatcatgatcaacccaacaacaacaacaacccatctgttaaatctgaagctagctcctcaagaatcaatcattactccatgttaatgagagccatccacaatactcaagaagctaacaacaacaacaatgacaacgtaagcgatgttgaagccatgaaggctaaaatcattgctcatcctcactactctaccctcctacaagcttacttggactgccaaaagattggagctccacctgatgtggttgatagaattacggcggcacggcaagactttgaggctcgacaacagcggtcaacaccgtctgtctctgcctcctctagagacccggagttagatcaattcatggaagcatactgtgacatgttggttaaatatcgtgaggagctaacaaggcccattcaggaagcaatggagtttatacgtcgtattgaatctcagcttagcatgttgtgtcagagtcccattcacatcctcaacaatcctgatgggaagagtgacaatatgggatcatcagacgaagaacaagagaataacagcggaggggaaacagaattaccggaaatagacccgagggccgaagatcgggaactcaagaaccatttgctgaagaagtatagtggatacttaagcagtttgaagcaagaactatccaagaagaaaaagaaaggtaaacttcctaaagaagcacggcagaagcttctcacgtggtgggagttgcattacaagtggccatatccttctgagtcagagaaggtagcgttggcggaatcaacggggttagatcagaaacaaatcaacaattggttcataaaccaaagaaagcgtcactggaaaccatctgaagacatgcagttcatggtgatggatggtctgcagcacccgcaccacgcagctctgtacatggatggtcattacatgggtgatggaccttatcgtctcggtccataagacatccaaaagctttagccacataataacaacctctcgttgctttcttgttacaactcatgttttgaattcccctacatcagtttgctacttatagctttctttgttttcacgccttttgtaatgtcttatgtcgttcggggagtttgagacttcctagtcagaaatatcccttcattttattttctctttttttcggtgtattttgtttttttgttttctcactaatgtttttatttaataatgtgtgaaaggaaaatgctattctg

(19) United States
(12) Patent Application Publication
(10) Pub. No.: US 2010/0071083 A1
(43) Pub. Date: Mar. 18, 2010

(54) TEMPERATURE-DEPENDENT MEGANUCLEASE ACTIVITY

(76) Inventors: James J. Smith, Durham, NC (US); Derek Jantz, Durham, NC (US)

Correspondence Address: WILMERHALE/BOSTON, 60 STATE STREET, BOSTON, MA, 02109, US

(21) Appl. No.: 12/403,093

(22) Filed: Mar. 12, 2009

Related U.S. Application Data
(60) Provisional application No. 61/035,797, filed on Mar. 12, 2008.

(51) Int. Cl. A01H 5/00; C12N 15/82; C12N 15/10

(52) U.S. Cl. 800/278; 800/298; 435/440

(57) ABSTRACT

The invention relates to methods for the production of genetically modified plants using engineered meganucleases and elevated temperature and to genetically modified plants produced by such methods.

### REFERENCE TO RELATED APPLICATIONS

This application claims the benefit of U.S. Provisional Application No. 61/035,797, filed Mar. 12, 2008, the disclosure of which is incorporated by reference herein in its entirety.

### FIELD OF THE INVENTION

The invention relates to the field of molecular biology and recombinant nucleic acid technology. In particular, the invention relates to methods for the production of genetically modified plants using engineered meganucleases and elevated temperature and to genetically modified plants produced by such methods.

### BACKGROUND OF THE INVENTION

Genome engineering requires the ability to insert, delete, substitute and otherwise manipulate specific genetic sequences within a genome, and has numerous therapeutic and biotechnological applications. The development of effective means for genome modification remains a major goal in gene therapy, agrotechnology, and synthetic biology (Porteus et al. (2005), Nat. Biotechnol. 23: 967-73; Tzfira et al. (2005), Trends Biotechnol. 23: 567-9; McDaniel et al. (2005), Curr. Opin. Biotechnol. 16: 476-83). A common method for inserting or modifying a DNA sequence involves introducing a transgenic DNA sequence flanked by sequences homologous to the genomic target and selecting or screening for a successful homologous recombination event. Recombination with the transgenic DNA occurs rarely, but can be stimulated by a double-stranded break in the genomic DNA at the target site. Numerous methods have been employed to create DNA double-stranded breaks, including irradiation and chemical treatments. Although these methods efficiently stimulate recombination, the double-stranded breaks are randomly dispersed in the genome, which can be highly mutagenic and toxic. At present, the inability to target gene modifications to unique sites within a chromosomal background is a major impediment to successful genome engineering.

One approach to achieving this goal is stimulating homologous recombination at a double-stranded break in a target locus using a nuclease with specificity for a sequence that is sufficiently large to be present at only a single site within the genome (see, e.g., Porteus et al. (2005), Nat. Biotechnol. 23: 967-73). The effectiveness of this strategy has been demonstrated in a variety of organisms using chimeric fusions between an engineered zinc finger DNA-binding domain and the non-specific nuclease domain of the FokI restriction enzyme (Porteus (2006), Mol Ther 13: 438-46; Wright et al. (2005), Plant J. 44: 693-705; Urnov et al. (2005), Nature 435: 646-51). Although these artificial zinc finger nucleases stimulate site-specific recombination, they retain residual non-specific cleavage activity resulting from under-regulation of the nuclease domain and frequently cleave at unintended sites (Smith et al. (2000), Nucleic Acids Res. 28: 3361-9). Such unintended cleavage can cause mutations and toxicity in the treated organism (Porteus et al. (2005), Nat. Biotechnol. 23: 967-73).

A group of naturally-occurring nucleases which recognize 15-40 base-pair cleavage sites commonly found in the genomes of plants and fungi may provide a less toxic genome engineering alternative. Such “meganucleases” or “homing endonucleases” are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001), Nucleic Acids Res. 29(18): 3757-3774). The LAGLIDADG meganucleases with a single copy of the LAGLIDADG motif (“mono-LAGLIDADG meganucleases”) form homodimers, whereas members with two copies of the LAGLIDADG motif (“di-LAGLIDADG meganucleases”) are found as monomers. Mono-LAGLIDADG meganucleases such as I-CreI, I-CeuI, and I-MsoI recognize and cleave DNA sites that are palindromic or pseudo-palindromic, while di-LAGLIDADG meganucleases such as I-SceI, I-Anil, and I-DmoI generally recognize DNA sites that are non-palindromic (Stoddard (2006), Q. Rev. Biophys. 38: 49-95).

Natural meganucleases from the LAGLIDADG family have been used to effectively promote site-specific genome modification in plants, yeast, Drosophila, mammalian cells and mice, but this approach has been limited to the modification of either homologous genes that conserve the meganuclease recognition sequence (Monnat et al. (1999), Biochem. Biophys. Res. Commun. 255: 88-93) or to pre-engineered genomes into which a recognition sequence has been introduced (Rouet et al. (1994), Mol. Cell. Biol. 14: 8096-106; Chilton et al. (2003), Plant Physiol. 133: 956-65; Puchta et al. (1996), Proc. Natl. Acad. Sci. USA 93: 5055-60; Rong et al. (2002), Genes Dev. 16: 1568-81; Gouble et al. (2006), J. Gene Med. 8(5):616-622).

Systematic implementation of nuclease-stimulated gene modification requires the use of genetically engineered enzymes with customized specificities to target DNA breaks to existing sites in a genome and, therefore, there has been great interest in adapting meganucleases to promote gene modifications at medically or biotechnologically relevant sites (Porteus et al. (2005), Nat. Biotechnol. 23: 967-73; Sussman et al. (2004), J. Mol. Biol. 342: 31-41; Epinat et al. (2003), Nucleic Acids Res. 31: 2952-62).

I-CreI is a member of the LAGLIDADG family which recognizes and cuts a 22 base-pair recognition sequence in the chloroplast chromosome, and which presents an attractive target for meganuclease redesign. The naturally-occurring enzyme is a homodimer in which each monomer makes direct contacts with 9 base pairs in the full-length recognition sequence. Genetic selection techniques have been used to modify the wild-type I-CreI cleavage site preference (Sussman et al. (2004), J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58). More recently, a method of rationally-designing mono-LAGLIDADG meganucleases was described which is capable of comprehensively redesigning I-CreI and other such meganucleases to target widely-divergent DNA sites, including sites in mammalian, yeast, plant, bacterial, and viral genomes (WO 2007/047859).

The agriculture industry is in particular need of a facile technology to target the insertion or removal of nucleic acids from the genomes of crop species. Although several methods have been disclosed for the production of engineered meganucleases that can, in principle, be used to target DNA breaks to predetermined sites in a genome, no method has been disclosed which enables the use of such engineered meganucleases for the genetic modification of plants. Previously disclosed methods concerning the use of meganucleases in plants (e.g., WO 03/004659, WO 06/032426, WO/2006/105946, and WO/2005/049842) enable the use of natural meganucleases and claim, by extension, the use of engineered meganucleases for similar purposes. The present disclosure distinguishes naturally-occurring and engineered meganucleases with respect to their DNA cleaving properties and provides unique methods by which engineered meganucleases can be made to function inside of a plant cell. Specifically, meganucleases which have been re-engineered with respect to DNA cleavage specificity have decreased cleavage activity relative to the naturally-occurring meganucleases from which they are derived (FIG. 1). This is particularly true at temperatures below 30° C. Thus, the present invention provides methods for the use of meganucleases to modify the genomes of plant cells which are unique to meganucleases which have been engineered with respect to DNA-cleavage specificity. Specifically, the present invention relates to the use of elevated temperature to stimulate the production of double-strand DNA breaks in a plant genome using engineered meganucleases.

### SUMMARY OF THE INVENTION

The present invention is a method for the production of genetically modified plants using engineered meganucleases. It relates to the use of elevated growth temperature to stimulate meganuclease-induced DNA breaks for the targeted insertion or removal of nucleic acids from the genome of a plant.

In one aspect, the invention provides a method for producing a genetically modified plant using an engineered meganuclease comprising: (a) introducing an engineered meganuclease or a construct encoding an engineered meganuclease into a plant cell; (b) introducing an exogenous sequence of interest into the plant cell; and (c) incubating the plant cell at a temperature of about 30° C. to about 44° C. for a period of about 1 minute to about 4 days, wherein the exogenous sequence of interest is inserted into the genome of the plant cell.

In some embodiments, the construct encoding the engineered meganuclease and the exogenous sequence of interest are part of the same DNA molecule.

In some embodiments, the exogenous sequence of interest comprises a gene.

In another aspect, the invention provides a method for inactivating an endogenous gene of interest in a plant using an engineered meganuclease comprising: (a) providing a plant cell comprising an endogenous gene of interest; (b) introducing an engineered meganuclease or a construct encoding an engineered meganuclease into the plant cell; and (c) incubating the plant cell at a temperature of about 30° C. to about 44° C. for a period of about 1 minute to about 4 days, wherein the engineered meganuclease cleaves the endogenous gene of interest and thereby inactivates it.

In still another aspect, the invention provides a method for excising an endogenous sequence of interest from a plant genome comprising: (a) providing a plant cell comprising an endogenous sequence of interest its genome; (b) introducing one or two engineered meganucleases or one or two constructs encoding one or two engineered meganucleases into the plant cell; and (c) incubating the plant cell at a temperature of about 30° C. to about 44° C. for a period of about 1 minute to about 4 days, wherein the one or two engineered meganucleases cleave a pair of DNA sites flanking the endogenous sequence of interest and the endogenous sequence of interest is excised from the plant genome.

In some embodiments, the plant cell is incubated a temperature of about 36° C. to about 44° C., about 30° C. to about 36° C., or about 38° C. to about 44° C.

In some embodiments, the plant cell is incubated for a period of about 1 hour to about 4 days. In other embodiments, the plant cell is incubated for a period of about 5 minutes to 1 about 1 hr, about 1 hour to about 12 hours, about 12 hours to about 24 hours, about 1 day to about 2 days, or about 2 days to about 4 days.

In some embodiments, the engineered meganuclease is derived from I-CreI.

In another aspect, the invention provides a method for producing a genetically modified plant using an engineered meganuclease comprising: (a) introducing an engineered meganuclease or a construct encoding an engineered meganuclease into a plant cell; (b) introducing an exogenous sequence of interest into the plant cell; and (c) raising the temperature of the transformed plant cell for a period of about 1 minute to about 4 days, wherein the exogenous sequence of interest is inserted into the genome of the plant cell.

In some embodiments, the temperature of the transformed plant cell raised by at least 5° C. or by at least 10° C.

In some embodiments, the temperature of the transformed plant cell is raised for a period of about 1 hour to about 4 days.

In some embodiments, the engineered meganuclease is derived from I-CreI.

In another aspect, the invention provides modified plant produced by any of the methods above and a descendant thereof.

### BRIEF DESCRIPTION OF THE FIGURES

FIG. 1: The effects of temperature on in vitro cleavage of DNA using engineered meganucleases. Three engineered meganucleases, BRP12-SC (SEQ ID NO: 4), MEG1, and MEG2, produced in accordance with WO 2007/047859 were evaluated for in vitro cleavage of a plasmid harboring the meganuclease recognition sequence for each at either 25° C. (dashed plots) or 37° C. (solid plots). A) 0.25 picomoles of plasmid substrate (linearized with XmnI) were incubated with 2.5 picomoles of purified meganuclease in 25 microliters of SA buffer (25 mM Tris, pH. 8.0; 100 mM NaCl; 1 mM EDTA; 5 mM MgCl₂) at the indicated temperature for the indicated length of time. Reactions were then stopped with the addition of 0.2% SDS and 1 unit of proteinase K and visualized by gel electrophoresis. The percentage of plasmid substrate cleaved was quantified using the ImageJ program and plotted as a function of incubation time. B) 0.25 picomoles of plasmid substrate (linearized with XmnI) were incubated with 5 microliters of the indicated concentration of purified meganuclease in 25 microliters of SA buffer (25 mM Tris, pH. 8.0; 100 mM NaCl; 1 mM EDTA; 5 mM MgCl₂) at the indicated temperature for 2 hours. Reactions were then stopped with the addition of 0.2% SDS and 1 unit of proteinase K and visualized by gel electrophoresis. The percentage of plasmid substrate cleaved was quantified using the ImageJ program and plotted as a function of meganuclease concentration. Both in vitro cleavage assays indicate a significant loss of engineered meganuclease activity at 25° C. relative to 37° C.

FIG. 2: Engineered meganucleases function inside plant cells provided transformed plants are incubated at elevated temperature. A) Schematic of a T-DNA that was stably integrated into the Arabidopsis thaliana genome as described in Example 1. In this T-DNA construct, a codon-optimized gene encoding the BRP2 meganuclease (SEQ ID NO: 1), (meganuclease), is under the control of a Hsp70 promoter (HSP) and a NOS terminator (TERM). A pair of BRP2 recognition sequences (Site1, Site2) are housed adjacent to the terminator separated by 7 base pairs containing a PstI restriction enzyme site (PstI). A kanamycin resistance marker (Kan) is also housed on the T-DNA to allow selection for stable transformants. B) The expected product following BRP2 meganuclease cleavage of Site1 and Site2 showing loss of the intervening 7 base pair fragment and PstI restriction site. Arrows show the location of PCR primers used to screen for cleavage of the T-DNA. C) Genomic DNA was isolated from the leaves of Arabidopsis seedlings stably transformed with the T-DNA diagrammed in (A) before and after a 2 hour “heat-shock” at 40° C. (as described in Example 1) and was added to PCR reactions using the primers shown in (B). PCR reactions were digested with PstI and visualized by gel electrophoresis. C: control lane lacking PstI. 44, 45, and 46: PCR samples from three representative plants showing nearly complete digestion by PstI in samples taken prior to heat shock (−lanes) and very little digestion by PstI in samples taken after heat-shock (+lanes). These results indicate that the BRP2 meganuclease was able to cleave the BRP2 recognition sequence primarily in cells exposed to elevated temperature. D) Heat-shocked T1 generation plants from (C) were self-pollinated and seeds from these crosses were grown into T2 generation seedlings on media containing kanamycin. Genomic DNA was isolated from these seedlings and subject to PCR and PstI digest as in (C). This analysis was performed for 10 T2 generation plants which show varying degrees of loss of the PstI site. In particular, plants 4 and 6 (arrows) show a complete loss of the PstI site, indicating that these plants are genetically uniform. These results indicate that heat-stimulated engineered meganucleases are active in germ-line tissue and can be used to generate uniformly genetically-modified plants.

FIG. 3: Marker gene excision using a heat-stimulated engineered meganuclease. A) T-DNA construct used to stably transform Arabidopsis thaliana as in FIG. 2a except the 7 base pair region intervening BRP2 recognition sequences in FIG. 2a has been replaced by a ˜1000 base pair basta resistance marker (BAR). B) The expected product following BRP2 meganuclease cleavage of the T-DNA in (A). C) Genomic DNA was isolated from 8 Arabidopsis seedlings stably transformed with the T-DNA diagrammed in (A) before and after a 2 hour heat-shock at 40° C. (see Example 2). PCR analysis of the genomic DNA samples using the primers shown in (B) (arrows) shows the presence of primarily a ˜1200 base pair PCR product (T-DNA with BAR gene) prior to heat-shock (−lanes) and the appearance of a ˜300 base pair product (T-DNA without BAR gene) following heat-shock. All PCR products were cloned and sequenced to verify sequence identity. These results that a marker gene can be efficiently excised from an integrated transgene using engineered meganucleases. They further indicate that such marker gene excision is significantly stimulated by incubation at elevated temperature.

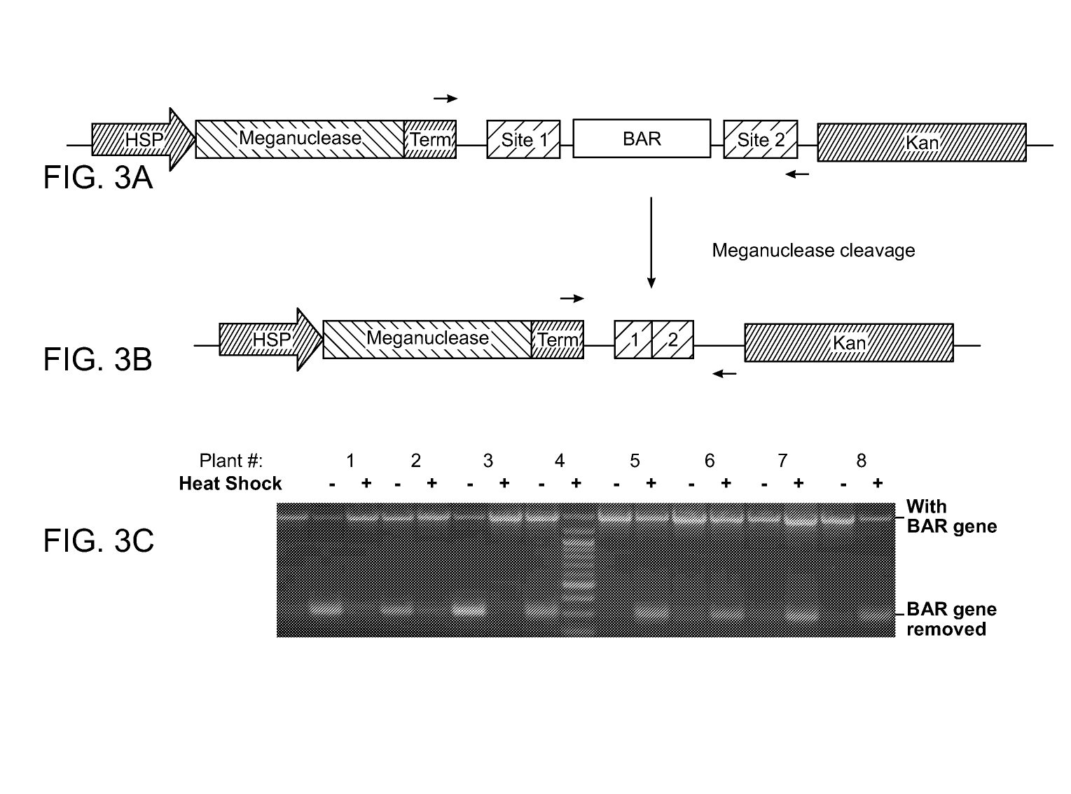

FIG. 4: Heat stimulated engineered meganucleases can target the mutation of a native plant gene. A T-DNA carrying a codon-optimized BRP12-SC gene (SEQ ID NO: 8) was used to stably transform Arabidopsis thaliana. The T-DNA was identical in structure to that diagrammed in FIG. 2a, except that it encoded the BRP12-SC meganuclease (SEQ ID NO: 4) instead of the BRP2 meganuclease. Genomic DNA was isolated from stable transformants before and after a 2 hour heat-shock at 40° C. (as described in Example 3). The DNA samples were then added to PCR reactions using primers to amplify a ˜1000 base pair fragment from the Arabidopsis KNAT1 gene (SEQ ID NO: 11) containing the BRP12-SC meganuclease recognition sequence. PCR products were then digested with XbaI to determine which had mutations introduced at the BRP12-SC meganuclease site. It was found that all PCR products retained the XbaI site prior to heat shock (lane 1) whereas a significant percentage of PCR products lost the XbaI site following heat shock (lane 2.) XbaI-resistant PCR products from lane 2 were cloned and sequenced and found to contain a variety of deletions at the BRP-12SC meganuclease recognition site.

### DETAILED DESCRIPTION OF THE INVENTION

### 1.1 Introduction

Methods for incorporating transgenes into the genomes of plant cells are well known in the art (e.g. Agrobacterium-mediated transformation, particle-bombardment, biolistic injection, “whiskers” transformation, and lipofection). These methods, however, integrate transgenes at more-or-less random locations in the genome. The ability to target transgene insertion and/or the deletion of DNA at discreet locations in the plant genome has numerous advantages over these existing methods. First, it enables modifications to be made in a region of the genome with known gene expression and/or heritability characteristics. Second, it enables the repeated targeting of trait genes to the same genomic locus, which will accelerate regulatory approval of subsequent genetically modified crop products following a first approval. Third, it enables plant genes to be knocked-out with high precision and efficiency. Fourth, it enables nucleic acids which were previously integrated into a genome (such as herbicide resistance genes) to be removed prior to regulatory submission. Lastly, it enables multiple genes to be inserted adjacent to one another in the same region of the genome so that they are genetically linked and will consistently co-segregate throughout subsequent breedings.

The use of rare-cutting homing endonucleases (“meganucleases”) to insert or remove DNA from the genome of a plant has been disclosed previously (e.g. WO 03/004659, WO 06/032426, WO/2006/105946, and WO/2005/049842). In particular, WO/2005/049842 discloses a method for the targeted insertion of a sequence of interest at the site of a meganuclease-induced DNA break using homologous recombination. Although these earlier inventions claim, broadly, the use of site-specific endonucleases to target plant genome modification, they specifically enable the use of naturally occurring meganucleases for this purpose. In each case, these earlier inventions are reduced to practice using the natural meganuclease I-SceI from Saccharomyces cerevisiae. Because the recognition sequence for I-SceI is relatively long (18 base pairs) this sequence occurs very rarely in nature and is unlikely to occur at a genomic region of interest purely by chance. The same can be said of all naturally-occurring meganucleases. As a consequence, earlier inventions were reduced to practice by first inserting a meganuclease (i.e. I-SceI) recognition sequence into the plant genome and subsequently targeting the modification of that introduced sequence using the corresponding meganuclease.

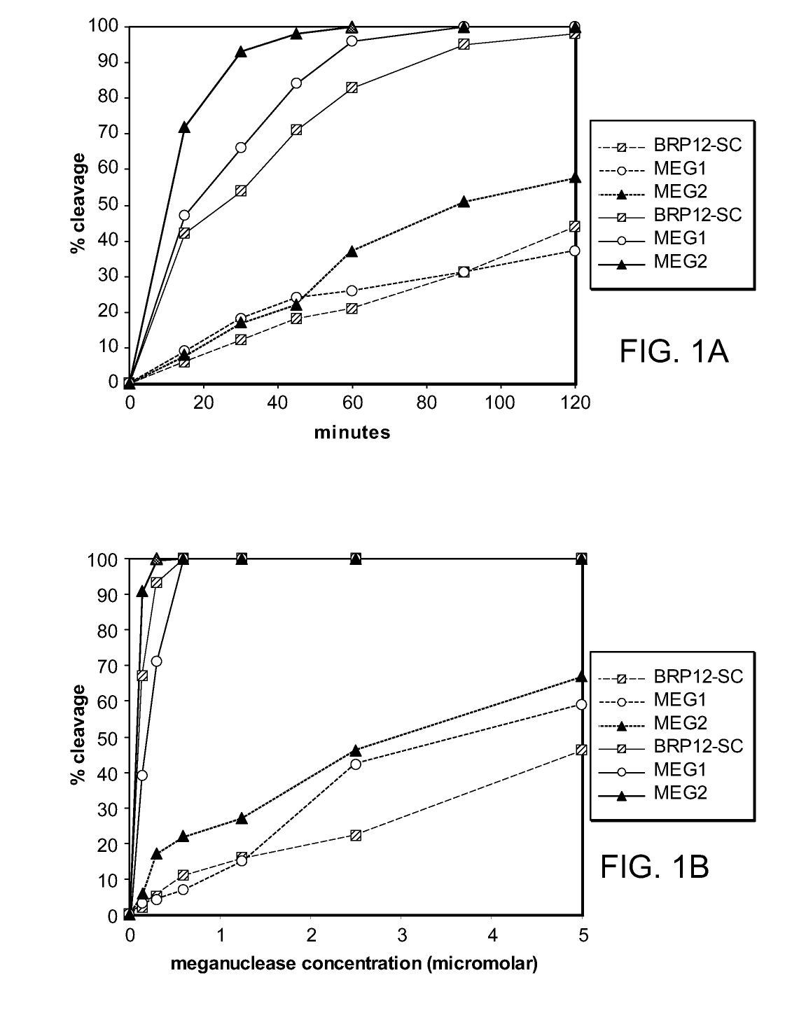

Several methods have been described which enable the production of engineered meganucleases with altered DNA-recognition properties (Sussman et al. (2004), J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58; WO 2007/047859). In particular, WO 2007/047859 describes methods for the structure-based engineering of meganucleases derived from the naturally-occurring meganuclease I-CreI. These engineered meganucleases can be made to recognize and cut pre-determined 22 base pair DNA sequences found in the genomes of plants, animals, fungi, bacteria, or yeast. Engineered meganucleases produced using the method described in WO 2007/047859 have been evaluated in plant cells (see Examples) and are shown to be capable of targeting genetic modifications to preexisting, natural locations in the genome. The requirements for producing such modifications using an engineered meganuclease, however, are shown to differ substantially from the requirements for doing so using a natural meganuclease. Specifically, engineered meganucleases are far more sensitive to reduced temperature than are their natural counterparts (FIG. 1). As a consequence, it is necessary to elevate plant growth temperature, at least transiently, to stimulate the activity of an engineered meganuclease inside of a plant cell. Disclosed methods which do not incorporate elevated temperature are, therefore, not enabled with respect to engineered meganucleases. The present invention is a method for stimulating the activity of an engineered meganuclease inside of a plant cell using elevated temperature. Thus, in certain embodiments, the invention provides methods for stimulating engineered meganuclease activity. In other embodiments, the invention provides methods for using heat-stimulated engineered meganucleases to modify the genomes of plant cells. In other embodiments, the invention provides transgenic plants and plant cells produced using such methods.

### 1.2 References and Definitions

The patent and scientific literature referred to herein establishes knowledge that is available to those of skill in the art. The issued U.S. patents, allowed applications, published foreign applications, and references, including GenBank database sequences, that are cited herein are hereby incorporated by reference to the same extent as if each was specifically and individually indicated to be incorporated by reference.

As used herein, the term “plant cell” refers to a protoplast, an intact cell isolated from a plant, a cell in plant tissue culture, or a cell inside a plant.

As used herein, the term “about” means within a range of −5% to +5% of the value that follows.

As used herein “inserted” means stably integrated, for example, stably integrated in to a genome.

As used herein “inactivate” means reducing activity (e.g., of a gene) by at least 10-fold. In some instances the activity can also be reduced by at least 100-fold or at least 1000-fold.

As used herein, the term “derived from” when used in context of a meganuclease means engineered from, for example, engineered from a certain naturally-occurring meganuclease.

As used herein, the term “meganuclease” refers to a naturally-occurring endonuclease that binds double-stranded DNA at a recognition sequence that is greater than 12 base pairs. Naturally-occurring meganucleases can be monomeric (e.g., I-SceI) or dimeric (e.g., I-CreI). The term meganuclease, as used herein, can be used to refer to monomeric meganucleases, dimeric meganucleases, or to the monomers which associate to form a dimeric meganuclease. The term “homing endonuclease” is synonymous with the term “meganuclease.”

As used herein, the term “engineered meganuclease” means a non-naturally occurring meganuclease that has been modified relative to a wild-type or a naturally-occurring meganuclease with respect to its DNA sequence recognition. Engineered meganucleases differ from the wild-type or naturally-occurring meganucleases in their amino acid sequence or primary structure, and may also differ in their secondary, tertiary or quaternary structure. In addition, engineered meganucleases differ from wild-type or naturally-occurring meganucleases in recognition sequence-specificity and/or cleavage activity. Engineered meganucleases may also differ from the wild-type or naturally-occurring meganucleases in their ability to dimerize (e.g., single-chain heterodimers may be produced by linking together a pair of individual subunits derived from I-CreI).

As used herein, with respect to a protein, the term “recombinant” means having an altered amino acid composition as a result of the application of genetic engineering techniques to nucleic acids which encode the protein, and cells or organisms which express the protein. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous different host, is not considered recombinant.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the term “wild-type” refers to any naturally-occurring form of a meganuclease. The term “wild-type” is not intended to mean the most common allelic variant of the enzyme in nature but, rather, any allelic variant found in nature. Wild-type meganucleases are distinguished from recombinant or non-naturally-occurring meganucleases.

As used herein, the term “recognition sequence half-site” or simply “half site” means a nucleic acid sequence in a double-stranded DNA molecule which is recognized by a monomer of a mono-LAGLIDADG meganuclease or by one LAGLIDADG subunit of a di-LAGLIDADG meganuclease.

As used herein, the term “recognition sequence” refers to a pair of inverted half-sites separated by four base pairs, which is bound and cleaved by either a mono-LAGLIDADG meganuclease dimer or a di-LAGLIDADG meganuclease monomer. In the case of I-CreI, the recognition sequence half-site of each monomer spans 9 base pairs, and the two half-sites are separated by four base pairs, designated N₁ through N₄, which are not recognized specifically. Thus, the combined recognition sequence of the I-CreI meganuclease dimer normally spans 22 base pairs, including the 9 base pairs 5′ of the central N₁-N₄ bases on the sense strand, which are designated −9 through −1, the central N₁-N₄ base pairs, and the 9 base pairs 3′ of the central N₁-N₄ bases on the sense strand, which are designated −1 through −9.

As used herein, the term “specificity” means the ability of a meganuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific meganuclease is capable of cleaving only one or a very few recognition sequences.

As used herein, the term “palindromic” refers to a recognition sequence consisting of inverted repeats of identical half-sites. In this case, however, the palindromic sequence need not be palindromic with respect to the four central nucleotide pairs, which are not contacted by the enzyme. In the case of dimeric meganucleases, palindromic DNA sequences are recognized by homodimers in which the two monomers make contacts with identical half-sites.

As used herein, the term “pseudo-palindromic” refers to a recognition sequence consisting of inverted repeats of non-identical or imperfectly palindromic half-sites. In this case, the pseudo-palindromic sequence not only need not be palindromic with respect to the four central nucleotide pairs, but also can deviate from a palindromic sequence between the two half-sites. Pseudo-palindromic DNA sequences are typical of the natural DNA sites recognized by wild-type homodimeric meganucleases in which two identical enzyme monomers make contacts with different half-sites.

As used herein, the term “non-palindromic” refers to a recognition sequence composed of two unrelated half-sites of a meganuclease. In this case, the non-palindromic sequence need not be palindromic with respect to either the four central nucleotide pairs or the two monomer half-sites. Non-palindromic DNA sequences are recognized by either highly degenerate meganucleases (e.g., I-CeuI) or by heterodimers of meganuclease monomers that recognize non-identical half-sites.

As used herein, the term “activity” refers to the rate at which a meganuclease of the invention cleaves a particular recognition sequence. Such activity is a measurable enzymatic reaction, involving the hydrolysis of phosphodiester bonds of double-stranded DNA. The activity of a meganuclease acting on a particular DNA substrate is affected by the affinity or avidity of the meganuclease for that particular DNA substrate which is, in turn, affected by both sequence-specific and non-sequence-specific interactions with the DNA.

As used herein, the term “meganuclease recognition site” refers to a region of a plant genome containing a meganuclease recognition sequence. By practicing the invention, an engineered meganuclease can be made to recognize a meganuclease recognition sequence at a meganuclease recognition site to modify the genome in the region of the meganuclease recognition site.

As used herein, the term “heat-stimulated engineered meganuclease” refers to an engineered meganuclease which is expressed in a plant cell or a plant for the purpose of cutting and modifying a meganuclease site wherein said plant cell or plant is grown at elevated temperature in accordance with the invention.

### 2. Heat-Stimulation of Engineered Meganucleases

Engineered meganucleases are more sensitive to reduced temperature than are wild-type meganucleases. FIG. 1 shows the in vitro cleavage activity of three engineered meganucleases with altered DNA sequence recognition produced in accordance with WO 2007/047859. The engineered meganucleases cut their respective meganuclease recognition sequences with high levels of activity at 37° C. but are significantly less active at 25° C. Because plant transformation and growth are typically performed at temperatures below 30° C., it is reasonable to believe that engineered meganucleases will not cleave DNA efficiently in a plant cell transformed and grown under standard conditions. Indeed, a pair of engineered meganuclease called “BRP2” and “BRP12-SC” produced in accordance with WO 2007/047859 (SEQ ID NO: 1, SEQ ID NO:4) were evaluated for function in transformed Arabidopsis thaliana and were found to be largely non-functional when expressed in plants grown at 25° C. (Examples 1-3). When plants transformed with the engineered meganucleases were “heat-shocked” for two hours at 40° C., however, the meganucleases cleaved their recognition sequences in the plant genome with high frequency. This was true of plants in which an artificial BRP2 meganuclease recognition sequence was pre-engineered into the genome (Examples 1, 2) as well as plants in which BRP12-SC recognized and cut an existing site in the Arabidopsis KNAT1 gene. Together, these observations lead us to conclude that elevated temperature is necessary for the high-frequency induction of site-specific DNA breaks in a plant genome using engineered meganucleases.

Thus, meganuclease-induced genome modification can be stimulated by elevating the growth temperature of a plant transformed with an engineered meganuclease gene for a period of at least one hour to a temperature of 30° C.-36° C. or, preferably, to a temperature of 36° C.-44° C. In the case of short incubation periods (1-4 hours) such incubation is preferably conducted by storing the transformed plant in a water bath at the elevated temperature. For incubations longer than 4 hours, the incubation is preferably conducted in a warm air incubator. Preferably the incubation at elevated temperature should not be conducted for longer than four days to avoid hindering plant growth. Moreover, the length of time for which a plant should be incubated at elevated temperature should be inversely proportional to the incubation temperature. Thus, the invention is optimally performed by storing the transformed plant for 1-4 hours in a water bath at 36° C.-44° C. or by storing the transformed plant for 4 hours to 4 days in a warm air incubator at 30° C.-36° C.

### 3. Methods of Producing Recombinant Plants Using Heat-Stimulated Engineered Meganucleases

Aspects of the present invention further provide methods for producing recombinant, transgenic or otherwise genetically-modified plant cells and plants using heat-stimulated engineered meganucleases. Thus, in one embodiment, heat-stimulated engineered meganucleases are used to cause a double-stranded break in a plant gene to mutate and inactivate gene expression. In another embodiment, heat-stimulated engineered meganucleases are used to cause a double-stranded break in a plant genome to allow for precise insertion(s) of a sequence of interest into that site by homologous recombination or non-homologous end joining. In another embodiment, heat-stimulated engineered meganucleases are used to cause a double-stranded break in a plant genome to excise a sequence of interest from the genome wherein the sequence of interest may or may not be transgene that was previously integrated into the genome using plant transformation techniques.

As used herein, the term “exogenous sequence of interest” means any DNA sequence that can be inserted into a plant genome. Exogenous sequences of interest will typically be genes that confer commercially valuable traits to crop species (e.g. genes that confer herbicide resistance, genes that confer insect resistance, genes that confer disease resistance, genes that confer drought resistance, genes that improve nutritional value, genes that improve yield or quality, and genes that affect plant fertility) as well as transcription regulation sequences (e.g. a promoter and transcription terminator) to control expression of the trait gene. These regulatory sequences include, but are not limited to, constitutive plant promoters such as the NOS promoter. The 35S promoter, or the UBI promoter, chemically-inducible gene promoters such as the dexamethasone-inducible promoter (see, e.g., Gremillon et al. (2004), Plant J. 37:218-228), and plant tissue specific promoters such as the LGC1 promoter (see, e.g., Singh et al. (2003), FEBS Lett. 542:47-52). Crop species include, but are not limited to, commercially valuable species such as corn, soybean, canola, sorghum, tobacco and tomato as well as research species such as Arabidopsis thaliana.

As used herein, the term “endogenous sequence of interest” means any DNA sequence that can be excised from a plant genome. The endogenous sequences of interest can be genes, open reading frames (ORFs), promoters, regulatory sequences, any fractions thereof, as well as any other endogenous sequences present in a plant genome.

As used herein, the term “endogenous gene of interest” means any endogenous plant gene that can be excised from a plant genome.

As used herein, the term “homologous recombination” refers to a natural, cellular process in which a double-stranded DNA-break is repaired using a homologous DNA sequence as the repair template (see, e.g. Cahill et al. (2006), Front. Biosci. 11:1958-1976). Thus, in some embodiments, a heat-stimulated engineered meganuclease is used to cleave a meganuclease recognition site in a plant cell and a sequence of interest flanked by DNA sequence with homology to the meganuclease recognition site is delivered into the cell and used as a template for repair by homologous recombination. The sequence of interest is thereby incorporated into the genome at the meganuclease recognition site.

As used herein, the term “non-homologous end-joining” refers to a natural, cellular process in which a double-stranded DNA-break is repaired by the direct joining of two non-homologous DNA segments (see, e.g. Cahill et al. (2006), Front. Biosci. 11:1958-1976). DNA repair by non-homologous end joining is error-prone and frequently results in the capture of exogenous DNA sequences at the site of repair joining (see, e.g. Salomon, et al. (1998), EMBO J. 17:6086-6095). This is particularly true of exogenous DNA sequences delivered by Agrobacterium tumefaciens. Thus, in certain embodiments, a heat-stimulated engineered meganuclease can be used to produce a double-stranded break at a meganuclease recognition site in a plant cell and a sequence of interest, which may or may not have homology to the meganuclease recognition site, can be delivered to the plant cell and be captured at the meganuclease recognition site by non-homologous end joining. The sequence of interest is, thereby, incorporated into the meganuclease recognition site.

As used herein, the term “meganuclease expression cassette” refers to a DNA sequence encoding an engineered meganuclease under the control of a promoter suitable for the expression of the meganuclease gene in a plant cell. Such promoters are known in the art and include, preferably, constitutive plant promoters such as the nopaline synthase (nos) promoter, the CaMV 35S promoter, or the plant ubiquitin (Ubi) promoter. In addition, for some embodiments, chemically-inducible gene promoters such as the dexamethasone-inducible promoter (see, e.g., Gremillon et al. (2004), Plant J. 37:218-228), and plant tissue specific promoters such as the LGC1 promoter (see, e.g., Singh et al. (2003), FEBS Lett. 542:47-52) may be used. Preferably, the meganuclease coding sequence will be optimized for expression in eukaryotic cells. Methods for codon optimization of a gene for expression in plant cells are known in the art. Also preferably, the meganuclease gene will be followed by a transcription terminator sequence such as the nopaline synthase (nos) terminator. Expression of a meganuclease gene in a plant cell can be verified using methods known in the art (e.g. Western Blot).

As used herein, the term “homologous donor cassette” refers to a DNA sequence comprising a sequence of interest flanked on one or, preferably, both sides by regions of homology to a meganuclease recognition site. The region(s) of homology will be at least 50 base pairs in length and will be, preferably 500-1000 base pairs in length. In preferable embodiments, the sequence of interest will be flanked on one side by 500-1000 bases that are identical or nearly identical to the DNA sequence immediately 5′ of the meganuclease recognition sequence (including the 5′ meganuclease recognition half-site) and will be flanked on the other side by 500-1000 bases that are identical or nearly identical to the DNA sequence immediately 3′ of the meganuclease recognition sequence (including the 3′ meganuclease recognition half-site). A homologous donor cassette may or may not be harbored on the same DNA molecule as a meganuclease expression cassette.

As a general matter, methods for delivering nucleic acids to plant cells are well known in the art and include Agrobacterium infection, PEG-mediated transformation of protoplasts (Omirulleh et al. (1993), Plant Molecular Biology, 21:415-428), desiccation/inhibition-mediated DNA uptake, electroporation, agitation with silicon carbide fibers, ballistic injection or microprojectile bombardment, and the like.

In some embodiments, the methods of the invention involve the genetic modification of a single plant cell or embryo which can be grown into a mature recombinant maize plant and give rise to progeny carrying the genetic modification of interest in its genome.

### 3.1 Methods for Inactivating a Gene in a Plant or Plant Cell Using Heat-Stimulated Engineered Meganucleases

Aspects of the invention allow for the use of engineered meganucleases to inactivate a gene in a plant cell or plant. Engineered meganucleases can be produced to cleave meganuclease recognition sites within the coding regions of plant genes. Such cleavage of a gene coding region frequently results in the deletion of DNA at the meganuclease recognition site following mutagenic DNA repair by non-homologous end joining (see, e.g., Example 3). Such mutations in the gene coding sequence are typically sufficient to inactivate the gene.

This method involves, first, the delivery of a meganuclease expression cassette to a plant cell or embryo using a suitable transformation method. For highest efficiency, it is desirable to link the meganuclease expression cassette to a selectable marker (i.e. an herbicide resistance marker) and select for successfully transformed cells in the presence of a selection agent (i.e. the corresponding herbicide). This approach will result in the integration of the meganuclease expression cassette into the genome, however, which may not be desirable if the plant is likely to require regulatory approval. In such cases, the meganuclease expression cassette (and linked selectable marker gene) may be segregated away in subsequent plant generations using conventional breeding techniques. Alternatively, plant cells may be initially be transformed with a meganuclease expression cassette lacking a selectable marker and may be grown on media lacking a selection agent. Under such conditions, a fraction of the treated cells will acquire the meganuclease expression cassette and will express the engineered meganuclease transiently without integrating the meganuclease expression cassette into the genome. Because it does not account for transformation efficiency, this latter transformation procedure requires that a greater number of treated cells be screened to obtain the desired genome modification.

Following delivery of a meganuclease expression cassette, plant cells are grown, initially, under conditions that are typical for the particular transformation procedure that was used. This typically means growing transformed cells or embryos on media at temperatures below 26° C., frequently in the dark. Such standard conditions should be used for a period of time, preferably 1-4 days, to allow the plant cell or embryo to recover from the transformation process. At any point following this initial recovery period, growth temperature can be raised to stimulate the activity of the engineered meganuclease to cleave and mutate the meganuclease recognition site. To recover plants in which the desired mutation is carried in as many cells as possible, it is preferable to induce meganuclease activity with elevated temperature as soon as is reasonable following recovery from the transformation procedure (i.e. before the transformant has undergone a large number of cell divisions and, so, will be mosaic with respect to mutations at the meganuclease recognition site).

### 3.2 Methods for Inserting a Sequence of Interest at a Meganuclease Recognition Site by Homologous Recombination

Aspects of the invention allow for the use of heat-stimulated engineered meganucleases to introduce a sequence of interest into a meganuclease recognition site by homologous recombination. Such methods require a meganuclease expression cassette and a homologous donor cassette. The two components, which may or may not be housed on the same DNA molecule are transformed simultaneously into a plant cell or embryo as described in 3.1. Alternatively, a meganuclease expression cassette may be transformed into a plant cell or embryo followed by subsequent transformation with a homologous donor cassette. In either case, transformed plant cells or embryos must be grown at elevated temperature as soon as is reasonable following recovery from the transformation procedure which introduced the homologous donor cassette (such recovery process is described in 3.1). This is because the homologous donor cassette is expected to persist in the plant cell nucleus for a limited length of time prior to being degraded or integrated into a random location in the genome. Thus, it is necessary to elevate temperature to induce the activity of the engineered meganuclease at an early point following transformation to “capture” the homologous donor cassette before it is lost. A typical gene insertion procedure will therefore, have the following sequence:
1. transform plant cells or embryos with a meganuclease expression cassette and a homologous donor cassette.
2. Recover transformed plant cells or embryos under conditions that are typical for the transformation procedure that was used for 1-4 days.
3. Elevate growth temperature to induce engineered meganuclease activity. The temperature should remain elevated for 1 hour to 5 days depending on the incubation method and temperature used, as described in (2) above.
4. Grow transformants into calli or seedlings and screen for the desired insertion event.

In a related embodiment, the efficiency of targeted insertion by homologous recombination using engineered meganucleases can be increased by using a pair of engineered meganucleases which cleave meganuclease recognition sites that exist in the genome of interest within 5,000 base pairs of one another. By cleaving the genome with a pair of engineered meganucleases, one ensures that a DNA break persists for a greater period of time because the region intervening the two meganuclease recognition sites is likely to be lost, resulting in DNA ends which are distal from one another. This embodiment is practiced as above with the following exceptions: first, plants must be transformed simultaneously with a pair of meganuclease expression cassettes which may or may not be housed on the same DNA molecule. Second, the homologous donor cassette should comprise a sequence of interest flanked on either side with a region of homology to the distal side of each meganuclease recognition site.

### 3.3 Methods for Inserting a Sequence of Interest at a Meganuclease Recognition Site by Non-Homologous End-Joining

Aspects of the invention allow for the use of heat-stimulated engineered meganucleases to introduce a sequence of interest into a meganuclease recognition site by non-homologous end joining This invention is practiced exactly as described for the case of insertion by homologous recombination in 3.2 above with the following exception: the homologous donor cassette, in this case, does not require a sequence with homology to the meganuclease recognition site. Because transgenes are preferentially captured at the site of DNA breaks in the genome, a sequence of interest can be integrated at a meganuclease recognition site through non-homologous end joining whether or not the sequence of interest has homology to the intended integration site. The final products produced by this type if integration will be variable, however, and will be less predictable than the product produced by insertion of a sequence of interest by homologous insertion.

In a related embodiment, the efficiency of targeted insertion by non-homologous end-joining using engineered meganucleases can be increased by using a pair of engineered meganucleases which cleave meganuclease recognition sites that exist in the genome of interest within 5,000 base pairs of one another. This embodiment is practiced as above with the following exception: plants must be transformed simultaneously with a pair of meganuclease expression cassettes which may or may not be housed on the same DNA molecule.

### 3.4 Methods for Excising a DNA Fragment from the Genome of a Plant Using Heat-Stimulated Engineered Meganucleases

For certain applications, it may be desirable to precisely remove a large DNA sequence from the genome of a plant. For example, it may be desirable to precisely remove an entire gene, gene cluster, or promoter. Such applications are possible using a pair of engineered meganucleases, each of which cleaves a meganuclease recognition site on either side of the intended deletion. This invention is practiced exactly as in 3.1 except plant cells or embryos are transformed with a pair of meganuclease expression cassettes which may or may not be housed on the same DNA molecule. In cases where the engineered meganuclease is derived from I-CreI (or any other mono-LAGLIDADG meganuclease), it is desirable to practice this invention using a single-chain derivative of each meganuclease to avoid the need to simultaneously express four genes inside of the cell.

In a related embodiment, heat-stimulated engineered meganucleases may be used to excise DNA from a plant genome that was previously integrated through plant transformation. For example, it is frequently desirable to transform commercially valuable crop species with genes encoding useful traits which are physically linked to selectable marker (i.e. herbicide resistance) genes so that transformed plants carrying the trait gene of interest can be selected for by growth under conditions which select for the marker gene. Moreover, it is frequently desirable to remove such marker genes prior to submission of a crop product for regulatory approval. This can be achieved by transforming plants with a trait expression cassette carrying a selectable marker gene in which the selectable marker gene is flanked by meganuclease recognition sequences for one or a pair of engineered meganucleases (see Example 2). At some point following this initial transformation event (possibly many generations later), the selectable marker gene is excised from the genome by exposure to heat-stimulated engineered meganuclease(s) such that only the desired trait gene is left behind in the genome. The process of marker excision may be practiced by transforming plant cells or embryos which carry the marker gene with a meganuclease expression cassette followed by growth at elevated temperature (as described in 3.1). Alternatively, a plant carrying the marker gene to be removed may be crossed with a second plant carrying a meganuclease expression cassette. The progeny from this cross may be grown at elevated temperature, as described, to induce meganuclease cutting of the meganuclease recognition sequences to excise the marker gene. Lastly, a meganuclease expression cassette comprising an engineered meganuclease gene under the control of an inducible promoter (e.g. a heat-shock promoter or a dexamethasone-inducible promoter) may be incorporated adjacent to the selectable marker gene, inside of the pair of meganuclease recognition sequences, such that meganuclease cleavage excises a larger fragment comprising both the selectable marker and the meganuclease expression cassette. Following the initial transformation of a plant cell or embryo with such a DNA construct and growth under conditions which select for the selectable marker gene, the fragment comprising the meganuclease expression cassette and the selectable marker can be excised by first growing the plant under conditions which activate expression of the inducible promoter and then elevating the growth temperature to stimulate the activity of the expressed meganuclease. By maintaining transformed plants under conditions which do not favor meganuclease expression and activity (i.e. reduced temperature and the absence of inducer), it is possible to carry plants for many generations prior to excising the selectable marker and meganuclease expression cassette.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

Elevated Temperature Stimulates the Activity of an Engineered Meganuclease in a Plant

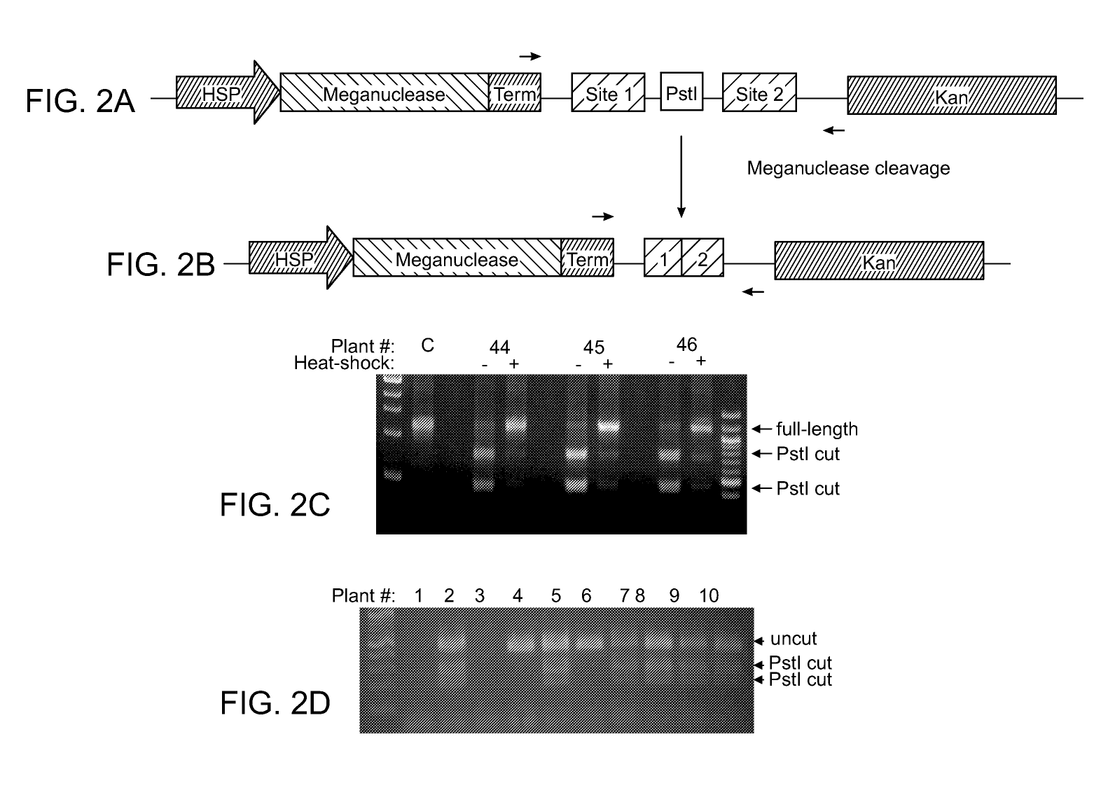

An engineered meganuclease called BRP2 (SEQ ID NO: 1) was produced using the method disclosed in WO 2007/047859. This meganuclease is derived from I-CreI and was engineered to recognize a DNA site that is not recognized by wild-type I-CreI (the BRP2 recognition sequence, SEQ ID NO: 3 and SEQ ID NO: 4). To facilitate nuclear localization of the engineered meganuclease, an SV40 nuclear localization signal (NLS, SEQ ID NO: 10) was added to the N-terminus of the protein. Conventional Agrobacterium-mediated transformation procedures were used to transform Arabidopsis thaliana with a T-DNA containing a codon-optimized BRP2 coding sequence (SEQ ID NO: 7). Expression of BRP2 meganuclease was under the control of a Hsp70 promoter and a NOS terminator. A pair of BRP2 recognition sequences were housed on the same T-DNA separated by 7 base pairs containing a PstI restriction enzyme site (FIG. 2a). BRP2 cutting of the pair of BRP2 recognition sequences in this construct was expected to excise the region intervening the recognition sequences and thereby remove the PstI restriction site (FIG. 2b).

Stably transformed Arabidopsis plants were produced by selection for a kanamycin resistance marker housed on the T-DNA. Genomic DNA was then isolated from the transformed plants (by leaf punch) before and the plants were wrapped in plastic wrap and incubated in a 40° C. water bath for 2 hours. Heat-shocked plants were then returned to soil and allowed to recover for 24 hours at 24° C. before genomic DNA was isolated a second time by leaf punch. Genomic DNA samples were then added to PCR reactions using primers to amplify the region of the T-DNA housing the meganuclease recognition sequences. PCR products were then digested with PstI and visualized by gel electrophoresis (FIG. 2c). It was found that, prior to heat-shock, the vast majority (>90%) of PCR samples retained the PstI site. After heat-shock, however, a large percentage of samples had lost the PstI site. PCR products lacking a PstI site were cloned into a pUC-19 plasmid and sequenced. 100% of sequenced clones had a precise deletion of the region intervening the two BRP2 cut sites (as diagrammed in FIG. 2b). These results indicate that elevated temperature is necessary for BRP2 meganuclease cleavage in plant cells.

Heat shocked plants were self-fertilized and next-generation plants were selected for T-DNA integration by selection on media containing kanamycin. Genomic DNA was isolated from these plants and subject to PCR and PstI digest as above (FIG. 2d). It was found that a high percentage of these T2 generation plants no longer had a PstI restriction site, indicating that the heat-stimulated BRP2 meganuclease was active in germ-line tissue.

### Example 2

### Marker Excision using a Heat-Stimulated Engineered Meganuclease

The experiment described in Example 1 was repeated with a T-DNA in which a basta resistance (BAR) gene was incorporated between the two BRP2 recognition sequences in place of the PstI site (FIG. 2a). In this experiment, BRP2 cleavage of the BRP2 recognition sequences flanking the BAR gene was expected to excise the BAR gene from the integrated T-DNA (FIG. 2b). PCR analysis of the T-DNA before and after a 2 hour heat-shock at 40° C. (as in Example 1) revealed that the BAR gene was efficiently excised from somatic leaf cell genomic DNA following the heat shock (FIG. 2c).

### Example 3

### Heat-Stimulated Engineered Meganucleases Cleave a Native Site in a Plant Genome

The engineered meganuclease BRP12-SC (SEQ ID NO: 4) was produced in accordance with WO 2007/047859 except that this meganuclease is a single-chain heterodimer. As discussed in WO/2007/047859, wild-type I-CreI binds to and cleaves DNA as a homodimer. As a consequence, the natural recognition sequence for I-CreI is pseudo-palindromic. The BRP12-SC recognition sequence (SEQ ID NO: 5, SEQ ID NO: 6), however, is non-palindromic. This necessitates the use of an engineered meganuclease heterodimer comprising a pair of subunits each of which recognizes one half-site within the full-length recognition sequence. In the case of BRP12-SC, the two engineered meganuclease monomers are physically linked to one another using an amino acid linker to produce a single-chain heterodimer. This linker comprises amino acids 166-204 (SEQ ID NO: 9) of BRP12-SC. The linker sequence joins an N-terminal meganuclease subunit terminated at L165 (corresponding to L155 of wild-type I-CreI) with a C-terminal meganuclease subunit starting at K204 (corresponding to K7 of wild-type I-CreI). The benefits of physically linking the two meganuclease monomers using this novel linker is twofold: first, it ensures that the meganuclease monomers can only associate with one another (heterodimerize) to cut the non-palindromic BRP12-SC recognition sequence rather than also forming homodimers which can recognize palindromic or pseudopalindromic DNA sites that differ from the BRP12-SC recognition sequence. Second, the physical linking of meganuclease monomers obviates the need to express two monomers simultaneously in the same cell to obtain the desired heterodimer. This significantly simplifies vector construction in that it only requires a single gene expression cassette. As was the case with the BRP2 meganuclease discussed in Examples 1 and 2, the BRP12-SC meganuclease has an SV40 nuclear localization signal (SEQ ID NO: 10) at its N-terminus.

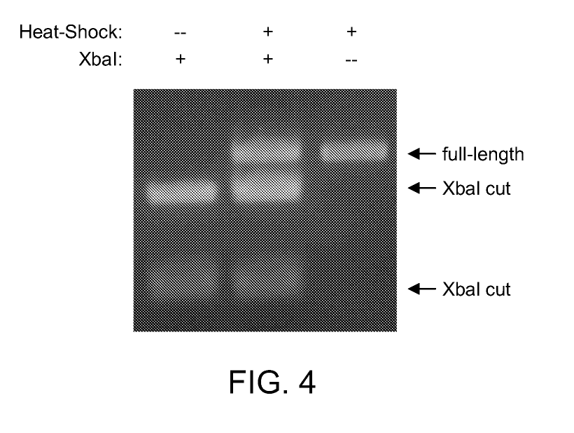

The BRP-SC recognition sequence (SEQ ID NO: 5 and SEQ ID NO: 6) is located in a native gene, KNAT1 (SEQ ID NO: 11, genbank accession # NM_{—}116884). Meganucleases cleavage of the BRP-SC recognition sequence in the KNAT1 gene and subsequent mutagenic repair by non-homologous end joining is expected to result in the introduction of mutations (primarily deletions) at the BRP-SC recognition site (as described for the embodiment in 3.1). Because the BRP-SC recognition sequence contains an XbaI restriction enzyme site (FIG. 4b), it was possible to screen for mutations at the BRP-SC recognition site by amplifying this region of the KNAT1 gene and digesting with XbaI. PCR products produced from mutated KNAT1 genes were expected to lose the XbaI site with some detectable frequency.

Arabidopsis were stably transformed by Agrobacterium-mediated transformation with a T-DNA harboring a codon-optimized BRP12-SC gene under the control of a Hsp70 promoter and a NOS terminator. Genomic DNA was isolated by leaf punch from T1 plants prior to a 2 hour heat-shock at 40° C. as described in Example 1. Plants were transferred to soil and allowed to recover for 24 hours at 24° C. before a second leaf punch was taken and genomic DNA was isolated from it. Genomic DNA samples were then added to a PCR reaction with primers to amplify a ˜800 base pair product from the KNAT1 gene containing the BRP12-SC recognition site. PCR products were then digested with XbaI and visualized by gel electrophoresis. PCR products produced from transformed plants prior to heat-shock had no detectable loss of the XbaI site. In contrast, XbaI failed to cut a portion of PCR products produced from heat-shocked plants, indicating that the BRP12-SC meganuclease recognition site was cleaved and mutated in the heat-shocked plants. XbaI-resistant PCR products from heat-shocked plants were cloned into a pUC-19 plasmid and sequenced. All clones had deletions at the BRP12-SC recognition site ranging in size from 2 base pairs to 514 base pairs. These results indicate that: 1) an engineered meganuclease can be used to mutate and inactivate a native gene in a plant, and 2) elevated temperature can be used to increase the cleavage activity of an engineered meganuclease inside of a plant cell.

1. A method for producing a genetically modified plant using an engineered meganuclease comprising:
(a) introducing an engineered meganuclease or a construct encoding an engineered meganuclease into a plant cell;
(b) introducing an exogenous sequence of interest into said plant cell; and
(c) incubating said plant cell at a temperature of about 30° C. to about 44° C. for a period of about 1 minute to about 4 days,
wherein said exogenous sequence of interest is inserted into the genome of said plant cell.

2. The method of claim 1 wherein the construct encoding said engineered meganuclease and said exogenous sequence of interest are part of the same DNA molecule.

3. The method of claim 1 wherein said exogenous sequence of interest comprises a gene.

4. A method for inactivating an endogenous gene of interest in a plant using an engineered meganuclease comprising:
(a) providing a plant cell comprising an endogenous gene of interest;
(b) introducing an engineered meganuclease or a construct encoding an engineered meganuclease into said plant cell; and
(c) incubating said plant cell at a temperature of about 30° C. to about 44° C. for a period of about 1 minute to about 4 days,
wherein said engineered meganuclease cleaves said endogenous gene of interest and thereby inactivates it.

5. A method for excising an endogenous sequence of interest from a plant genome comprising:
(a) providing a plant cell comprising an endogenous sequence of interest its genome;
(b) introducing one or two engineered meganucleases or one or two constructs encoding one or two engineered meganucleases into said plant cell; and
(c) incubating said plant cell at a temperature of about 30° C. to about 44° C. for a period of about 1 minute to about 4 days,
wherein said one or two engineered meganucleases cleave a pair of DNA sites flanking said endogenous sequence of interest and said endogenous sequence of interest is excised from the plant genome.

6. The method of any one of claims 1-5 wherein said plant cell is incubated a temperature of about 36° C. to about 44° C.

7. The method of any one of claims 1-6 wherein said plant cell is incubated a temperature of about 30° C. to about 36° C.

8. The method of any one of claims 1-5 wherein said plant cell is incubated a temperature of about 38° C. to about 44° C.

9. The method of any one of claims 1-8 wherein said plant cell is incubated for a period of about 1 hour to about 4 days.

10. The method of any one of claims 1-9 wherein said engineered meganuclease is derived from I-CreI.

11. A genetically modified plant produced by a method of any one of claims 1-10 and a descendant thereof.

12. A method for producing a genetically modified plant using an engineered meganuclease comprising:
(a) introducing an engineered meganuclease or a construct encoding an engineered meganuclease into a plant cell;
(b) introducing an exogenous sequence of interest into said plant cell; and
(c) raising the temperature of said transformed plant cell for a period of about 1 minute to about 4 days,
wherein said exogenous sequence of interest is inserted into the genome of said plant cell.

13. The method of claim 12 wherein the temperature of said transformed plant cell is raised by at least 5° C.

14. The method of claim 12 wherein the temperature of said transformed plant cell is raised by at least 10° C.

15. The method of any one of claims 12-14 wherein the temperature of said transformed plant cell is raised for a period of about 1 hour to about 4 days.

16. The method of any one of claims 12-15 wherein said engineered meganuclease is derived from I-CreI.

17. A genetically modified plant produced by a method of any one of claims 12-17 and a descendant thereof.
